(19) United States
(12) Patent Application Publication
(10) Pub. No.: US 2019/0200878 A1
(43) Pub. Date: Jul. 4, 2019

(54) CARDIAC MONITORING SYSTEM AND METHOD

(71) Applicant: KONINKLIJKE PHILIPS N.V., EINDHOVEN (NL)

(72) Inventor: Jin WANG, Shanghai (CN)

(21) Appl. No.: 16/092,889

(22) PCT Filed: Apr. 11, 2017
(86) PCT No.: PCT/EP2017/058600

§ 371 Date: Oct. 11, 2018

(30) Foreign Application Priority Data
Apr. 13, 2016 (CN) PCT/CN2016/079178
May 17, 2016 (EP) 16169956.6

(51) Int. Cl. A61B 5/0205; A61B 5/00; A61B 5/11; A61B 5/02; A61B 5/08; A61B 5/0245

(52) U.S. Cl. CPC A61B 5/0205; A61B 5/746; A61B 5/1118; A61B 5/02028; A61B 5/7475; A61B 5/0245; A61B 5/7275; A61B 2562/0219; A61B 2505/09; A61B 5/0816

(57) ABSTRACT

A cardiac analysis system is for use during a cardiac rehabilitation exercise. The system monitors the user's heart rate, activity level and respiration frequency and thresholds are set for these parameters. The system then determines whether or not an exercise being undertaken is suitable for cardiac rehabilitation.

### FIELD OF THE INVENTION

This invention relates to a cardiac monitoring system and method, and in particular for use as part of a cardiac rehabilitation system and method.

### BACKGROUND OF THE INVENTION

Cardiovascular disease is the leading cause of death in many countries. Cardiac rehabilitation can reduce mortality by 12% to 34% based on some of the clinical studies carried out in western countries.

In the last ten years, the cardiac surgery volume in China has for example achieved over 10% year on year growth, and cardiovascular disease is becoming one of the biggest threats to Chinese residents. The same situation is also present in other industrialized nations.

A limited number of hospitals have cardiac rehabilitation units so that only a small fraction of eligible patients participate in cardiac rehabilitation programs.

Cardiac rehabilitation evaluation for example uses cardiopulmonary exercise testing. The patient performs one or more exercise tests on an ergometer. Following the test, the patient is given a so-called Borg score (a self-feeling score) as shown in the table below.

| Score | Self-feeling |
|---|---|
| 6 | Very relaxing |
| 7 | |
| 8 | |
| 9 | Very light |
| 10 | |
| 11 | Light |
| 12 | |
| 13 | A little tight |
| 14 | |
| 15 | Tight |
| 16 | |
| 17 | Very tight |
| 18 | |
| 19 | Very very tight |
| 20 | |

The exercise will be considered to be efficient for cardiovascular rehabilitation if the patient's score is in the range 11 to 14. Otherwise the exercise is stopped.

Existing home use cardiac rehabilitation solutions monitor exercise or other physical activity by monitoring the heart rate and activity level. However, the patient still needs to perform a self-feeling evaluation during the exercise, and then stop the exercise when the patient feels bad, since the key indicator of the cardiac functionality, e.g. heart rate, will be influenced by medication and may not therefore represent the real situation of the heart, if the medication is taken not long enough before the exercises.

There is therefore a need for a system that can evaluate the suitability of exercise for cardiac rehabilitation and thereby provide advice to a patient that the exercise should be stopped.

### SUMMARY OF THE INVENTION

The invention is defined by the claims.

According to examples in accordance with an aspect of the invention, there is provided a cardiac analysis system for use during cardiac rehabilitation exercise, comprising:

a first input for receiving an indication of a heart rate of a user on a cardiac medication;

a second input for receiving an indication of an activity level of the user of the system;

a third input for receiving an indication of the respiration frequency of the user of the system, the respiration frequency independent of the influence from the cardiac medication;

a memory for storing thresholds for each of the activity level, the heart rate and the respiration frequency for a particular user, wherein the thresholds define ranges of activity level, heart rate and respiration frequency corresponding to the recovery progress of the particular user; and

a controller for processing the indications received at the first to third inputs, comparing them with the thresholds, and determining whether or not the exercise being undertaken is suitable for cardiac rehabilitation.

This system enables automatic determination of whether an exercise is suitable for cardiac rehabilitation. The user of the system is typically a patient undergoing cardiac rehabilitation. By setting patient-specific thresholds, account can be taken of the medications the patient is taking, which may for example influence their heart rate. The system avoids the need for the patient repeatedly to perform self-assessment of their feeling of exertion during their rehabilitation exercises. Instead, the system derives this information from the threshold levels.

Ideally, all the key indicators would be independent of any influence from medications, so that the indicators only reflect how the heart is working. Unfortunately, some key indicators, including heart rate, may be influenced by medication. This is why the subjective Borg score is often used instead of an objective evaluation of physiological measurements. By using supplementary parameters which are independent of any medication influence, e.g. respiration rate, correction can be made for this influence, so that objective observations are able to replace a patient self-evaluation. The respiration frequency of the user is for example likely to be independent of medications taken by the patient, whereas medication may influence the heart rate. By combining parameters in this way, the system becomes more versatile and able to provide correct cardiac rehabilitation advice for a variety of patients on different medications.

If the heart rate, activity level and respiratory frequency value do not fall into a single set of predefined thresholds, the parameters may be given priority levels. For example, the heart rate may be dominant over the respiration frequency which may be dominant over the activity level. Alternatively, the parameters may be combined to form a single metric. Alternatively, manual selection of possible Borg scores by the user may be employed.

The system may comprise a fourth input for receiving an indication from the user of the user's feeling of the effort required by the exercise.

In this way, the user can also provide his or her own input. This may be part of a training cycle, during which the thresholds are initially set or it may be carried out during use of the system.

The controller may be adapted to update the thresholds in dependence on the fourth input. This provides a self-learning process.

This fourth input for example assists in reducing any ambiguity in the interpretation of the measured parameters. The user can input a score which represents their current feeling on a touch screen or other interactive input device during the exercise. The heart rate, activity level and respiratory frequency are then associated with the corresponding score. The rules based on the inputs can dynamically update the mapping table to a more personalized version, so that it will rarely happen that the measured parameters cannot be interpreted by a suitable mapping table.

The controller may be adapted to derive a Borg rating of the perceived exertion.

In this way, the system can set the internal thresholds based on a user's own Borg rating of the perceived exertion. By outputting the system-derived Borg score, the user of the system can judge if the conclusion seems correct or if the thresholds need to be updated further.

The thresholds may define four sub-categories of suitable exercise conditions corresponding to a Borg rating of perceived exertion score of 11, 12, 13 or 14. These are the Borg scores which are considered suitable for cardiac rehabilitation.

The controller may be adapted to provide an output warning when the exercise is not determined to be suitable for cardiac rehabilitation.

The user may for example be instructed to stop the exercise because it may present a danger.

The invention also provides a cardiac monitoring system comprising:

a cardiac analysis system as defined above;

a heart rate monitor;

a respiration monitor; and

an activity level monitor.

The system may receive inputs from remote sensors, or else a cardiac monitoring system may include the sensors required.

The activity level monitor may comprise an accelerometer.

Examples in accordance with a second aspect of the invention provide a cardiac analysis method for use during cardiac rehabilitation exercise of a subject, comprising:

storing thresholds for each of the activity level, the heart rate and the respiration frequency for a particular user, wherein the thresholds define safe ranges of activity level, heart rate and respiration frequency corresponding to the recovery progress of the particular user;

receiving an indication of a heart rate of the subject;

receiving an indication of an activity level of the subject;

receiving an indication of the respiration frequency of the subject; and

processing the indications received by comparing them with the thresholds, and thereby determining whether or not the exercise being undertaken is suitable for cardiac rehabilitation.

This method enables automatic determination of whether an exercise is suitable for cardiac rehabilitation of a subject, typically a patient with cardiovascular disease.

The method may comprise receiving an indication from the subject of the subject's own feeling of the effort required by the exercise and the controller may be adapted to update the thresholds in dependence on the indication of the subject's own feeling of the effort required by the exercise.

A Borg rating of the perceived exertion score may be derived. An output warning may be provided when the exercise is not determined to be suitable for cardiac rehabilitation.

The processing may be implemented at least partially in software.

### BRIEF DESCRIPTION OF THE DRAWINGS

Examples of the invention will now be described in detail with reference to the accompanying drawings, in which:

FIG. 1 shows a cardiac analysis system;

FIG. 2 shows a cardiac analysis method; and

FIG. 3 shows a general computer architecture suitable for implementing the controller of the system of FIG. 1.

### DETAILED DESCRIPTION OF THE EMBODIMENTS

The invention provides a cardiac analysis system for use during a cardiac rehabilitation exercise. The system monitors the user's heart rate, activity level and respiration frequency and thresholds are set for these parameters. The system then determines whether or not an exercise being undertaken is suitable for cardiac rehabilitation.

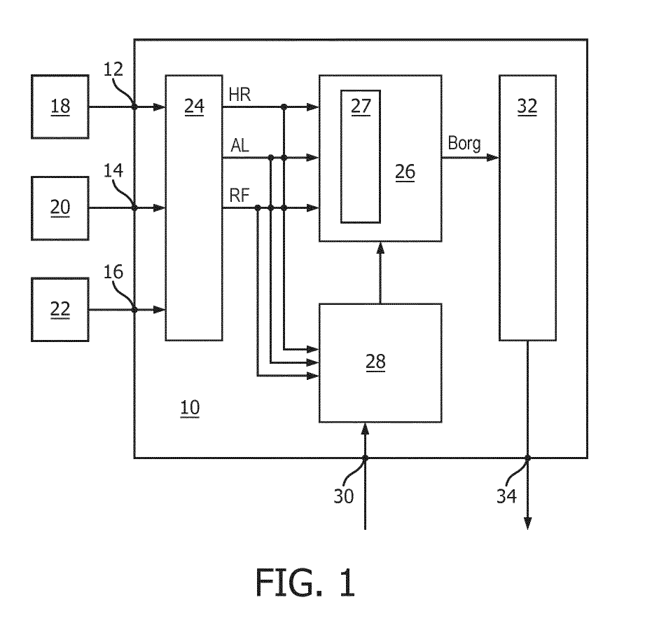

FIG. 1 shows a cardiac monitoring system system for use during a cardiac rehabilitation exercise. It comprises a cardiac analysis system 10 which has a first input 12 for receiving an indication of a heart rate of a user of the system, a second input 14 for receiving an indication of an activity level of the user of the system and a third input 16 for receiving an indication of the respiration frequency of the user of the system.

To provide the inputs to the analysis system, there is a heart rate monitor 18 such as an ECG sensor, an activity level monitor 20 such as an accelerometer and a respiration monitor 22 such as a respiration sensor.

These sensors are all well-known and there are many different alternative sensor types. For example, a PPG sensor may be used to derive both heart rate and respiration frequency. Respiration rate may instead be obtained using a chest belt which measures chest movement or a mask which measures breathing flow or flow direction. An activity level may be provided either by sensors associated with the user such as an accelerometer or pedometer, or by a monitoring system associated with a piece of exercise equipment being used by the user.

The monitor outputs are provided to a signal acquisition and processing unit 24 which derives the heart rate (HR), a metric of activity level (AL) and a respiration frequency (RF).

A controller 26 and associated memory 27 functions as a mapping module, and it stores thresholds for each of the activity level, the heart rate and the respiration frequency for a particular user. The thresholds define ranges of activity level, heart rate and respiration frequency for the particular user.

The mapping implemented by the controller 26 and memory 27 is dynamic and it is updated by a training module 28. The training module receives a fourth input 30 for receiving an indication from the user of the user's feeling of the effort required by the exercise.

In this way, the user provides input which is used as part of a training cycle, during which the thresholds in the memory 26 are initially set.

The table below shows an example of the mapping implemented by the memory 26 from threshold ranges for the heart rate, activity level and respiratory frequency, to a Borg score.

| | | Heart rate | Activity level from | Respiratory frequency |
|---|---|---|---|---|
| Score | Self-feeling | from ECG (HR) | accelerometer sensor (AL) | from respiratory sensor (RF) |
| 6 | Very | HR < HR_Th1 | AL < AL_Th1 | RF < RF_Th1 |
| 7 | relaxing | HR_Th1 < HR < HR_Th2 | AL_Th1 < AL < AL_Th2 | RF_Th1 < RF < RF_Th2 |
| 8 | | HR_Th2 < HR < HR_Th3 | AL_Th2 < AL < AL_Th3 | RF_Th2 < RF < RF_Th3 |
| 9 | Very light | HR_Th3 < HR < HR_Th4 | AL_Th3 < AL < AL_Th4 | RF_Th3 < RF < RF_Th4 |
| 10 | | HR_Th4 < HR < HR_Th5 | AL_Th4 < AL < AL_Th5 | RF_Th4 < RF < RF_Th5 |
| 11 | Light | HR_Th5 < HR < HR_Th6 | AL_Th5 < AL < AL_Th6 | RF_Th5 < RF < RF_Th6 |
| 12 | | HR_Th6 < HR < HR_Th7 | AL_Th6 < AL < AL_Th7 | RF_Th6 < RF < RF_Th7 |
| 13 | A little tight | HR_Th7 < HR < HR_Th8 | AL_Th7 < AL < AL_Th8 | RF_Th7 < RF < RF_Th8 |
| 14 | | HR_Th8 < HR < HR_Th9 | AL_Th8 < AL < AL_Th9 | RF_Th8 < RF < RF_Th9 |
| 15 | tight | HR_Th9 < HR < HR_Th10 | AL_Th9 < AL < AL_Th10 | RF_Th9 < RF < RF_Th10 |
| 16 | | HR_Th10 < HR < HR_Th11 | AL_Th10 < AL < AL_Th11 | RF_Th10 < RF < RF_Th11 |
| 17 | Very tight | HR_Th11 < HR < HR_Th12 | AL_Th11 < AL < AL_Th12 | RF_Th11 < RF < RF_Th12 |
| 18 | | HR_Th12 < HR < HR_Th13 | AL_Th12 < AL < AL_Th13 | RF_Th12 < RF < RF_Th13 |
| 19 | Very very | HR_Th13 < HR < HR_Th14 | AL_Th13 < AL < AL_Th14 | RF_Th13 < RF < RF_Th14 |
| 20 | tight | HR_Th14 < HR < HR_Th15 | AL_Th14 < AL < AL_Th15 | RF_Th14 < RF < RF_Th15 |

Heart rate thresholds are shown as HR_Th1 to HR_Th15. Activity level thresholds are shown as AL_Th1 to AL_Th15. Respiration frequency thresholds are shown as RF_Th1 to RF_Th15.

In this example, the thresholds divide the total parameter space into a continuous set of bands with no overlap. Thus, each heart rate, activity level and respiratory frequency has a one-to-one mapping to a Borg score. As explained below, this is only one option for implementing the thresholds.

The thresholds can initially be set by a doctor giving cardiac rehabilitation evaluation and exercise training, by inputting a Borg score to the fourth input 30. At the time the input is provided, the system is aware of the current heart rate, activity level and respiration frequency and can thus make an association. The controller 26 then updates the thresholds stored in the memory 27 in dependence on the fourth input 30.

The thresholds are defined according to the recovery progress of the user, which will vary during the cardiac rehabilitation, since the heart function is improving and can cope with higher standards gradually.

In addition, the fourth input 30 may be used by the patient during use of the system. This provides a self-learning process. Thus, each Borg score indication provided to the fourth input 30 is associated with the heart rate, activity level and respiration frequency at that time.

The output from the controller 26 is for example a Borg score. From this Borg score, the system can determine if the exercise being undertaken is suitable for cardiac rehabilitation.

An output device 32 provides an output 34 to the user, providing information about the suitability of the exercise for cardiac rehabilitation, for example an indication of whether or not the current monitored parameters correspond to a Borg score of 11 to 14.

Since the Borg score of 11 to 14 is of primary interest, a simplified set of thresholds may be defined as shown below.

| | | | | Respiratory |
|---|---|---|---|---|
| | | | Activity level | frequency |
| | | | from | from |
| | Self- | Heart rate | accelerometer | respiratory |
| Score | feeling | from ECG (HR) | sensor (AL) | sensor (RF) |
| 6 | Very | HR < HR_Th5 | AL < AL_Th5 | RF < RF_Th5 |
| 7 | relaxing | | | |
| 8 | | | | |
| 9 | Very | | | |
| 10 | light | | | |
| 11 | Light | HR_Th5 < HR < | AL_Th5 < AL < | RF_Th5 < RF < |
| | | HR_Th6 | AL_Th6 | RF_Th6 |
| 12 | | HR_Th6 < HR < | AL_Th6 < AL < | RF_Th6 < RF < |
| | | HR_Th7 | AL_Th7 | RF_Th7 |
| 13 | A little | HR_Th7 < HR < | AL_Th7 < AL < | RF_Th7 < RF < |
| | tight | HR_Th8 | AL_Th8 | RF_Th8 |
| 14 | | HR_Th8 < HR < | AL_Th8 < AL < | RF_Th8 < RF < |
| | | HR_Th9 | AL_Th9 | RF_Th9 |
| 15 | tight | HR_Th9 < HR | AL_Th9 < AL | RF_Th9 < RF |
| 16 | | | | |
| 17 | Very | | | |
| 18 | tight | | | |
| 19 | Very | | | |
| 20 | very | | | |
| | tight | | | |

This simplifies the data required.

This system enables automatic determination of whether an exercise is suitable for cardiac rehabilitation. By setting patient-specific thresholds both as part of a training procedure but also during using of the system, account can be taken of the medications the patient is taking, which may for example influence their heart rate.

The respiration frequency of the user is likely to be independent of any medications taken by the patient, whereas medication may influence the heart rate. Some medication can for example lower the heart rate, so that a different threshold is needed. A heart rate lowered by the medication may otherwise place the patient in danger. The patient may for example make use of the system at the same time within their cycle of medication taking so that the thresholds remain valid. The system may for example be adapted to remind the patient not to do exercise within a certain time of taking a particular medication.

It may of course happen that the current heart rate, activity level and respiration frequency do not all fall neatly into the threshold band for the same Borg score. A number of different approaches may then be adopted.

The parameters may be given priority levels. For example, the heart rate may be dominant over the respiration frequency which may be dominant over the activity level. This dominance may for example be used if there is only a small difference in Borg value. For example if the there are two adjacent Borg scores predicted by the measured parameters, then the one which is predicted by the heart rate may be chosen.

A greater level of divergence from a single score may indicate that more involved data processing is needed.

For example, there may be a weighted combination of the parameters to form a single metric. A more dominant parameter then has a greater weighting.

As part of a continued learning process, when ambiguity arises, the user may be prompted to give manual input (to the fourth input 30) indicating the actual Borg score. The controller can improve the way it interprets the data, and adjust thresholds accordingly.

There may be thresholds which are not continuous, but which overlap between categories. Thus, each heart rate, activity level and respiration frequency may map to two or three possible Borg scores. A best fit approach is then applied to the set of measured parameters to determine which single Borg category best fits the totality of the collected sensor data.

The user may use a touch screen or other interactive input device during the exercise to provide their input. The rules which are used to interpret the sensor inputs can thus dynamically update the mapping table to a more personalized version.

Based on the output 34 provided by the system, the user of the system can judge if the conclusion seems correct, or if the thresholds need to be updated. The output may include the determined Borg score at all times, and additionally a warning when the exercise is not determined to be suitable for cardiac rehabilitation. The user may for example be instructed to stop the exercise because it may present a danger.

An additional input to the system may comprise a camera for analyzing a facial expression or facial color of the user as a further indication of their level of exertion.

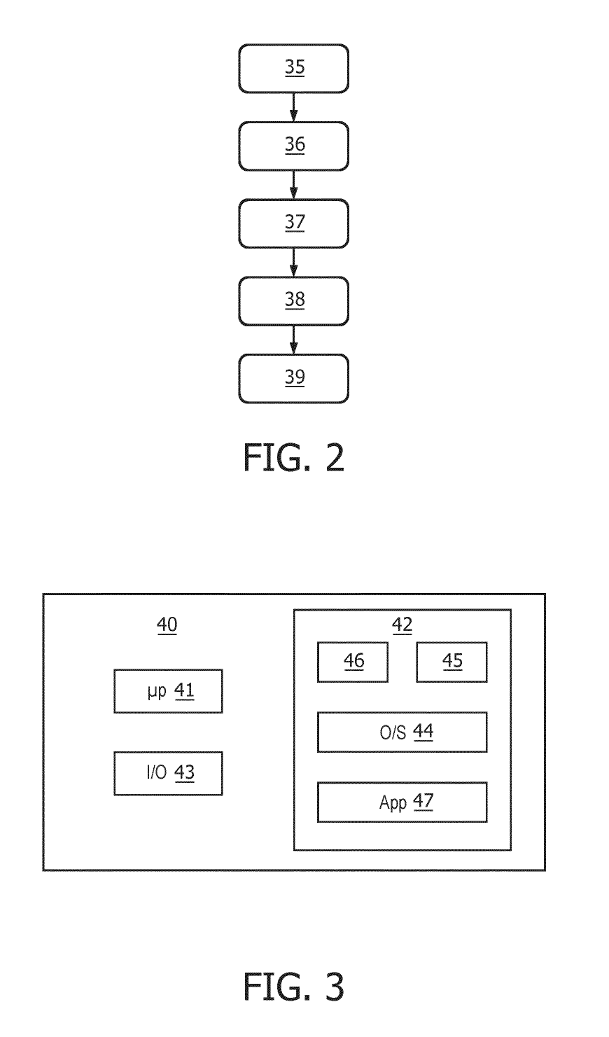

FIG. 2 shows a cardiac analysis method for use during cardiac rehabilitation exercise of a subject. The subject may be any person, but typically it is a patient with cardiovascular disease or damage. The method comprises, in step 35, storing thresholds for each of the activity level, the heart rate and the respiration frequency for a particular user, wherein the thresholds define ranges of activity level, heart rate and respiration frequency for the particular user.

In step 36, indications of a heart rate of the user, an activity level of the user and the respiration frequency of the user are received.

In step 37, the indications received are compared with the thresholds, and thereby it is determined whether or not the exercise being undertaken is suitable for cardiac rehabilitation.

In step 38, an indication is received from the user of the user's own feeling of the effort required by the exercise. This is used as part of a database training or recalibration operation. In particular, the thresholds may be updated dependence on the indication of the user's own feeling of the effort required by the exercise.

An output warning is provided in step 39 when the exercise is not determined to be suitable for cardiac rehabilitation.

The system described above makes use of a controller for processing the sensor data and applying and updating the thresholds stored memory.

FIG. 3 illustrates an example of a computer 40 for implementing the controller described above.

The computer 40 includes, but is not limited to, PCs, workstations, laptops, PDAs, palm devices, servers, storages, and the like. Generally, in terms of hardware architecture, the computer 40 may include one or more processors 41, memory 42, and one or more I/O devices 43 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 41 is a hardware device for executing software that can be stored in the memory 42. The processor 41 can be virtually any custom made or commercially available processor, a central processing unit (CPU), a digital signal processor (DSP), or an auxiliary processor among several processors associated with the computer 40, and the processor 41 may be a semiconductor based microprocessor (in the form of a microchip) or a microprocessor.

The memory 42 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 42 may incorporate electronic, magnetic, optical, and/or other types of storage media. Note that the memory 42 can have a distributed architecture, where various components are situated remote from one another, but can be accessed by the processor 41.

The software in the memory 42 may include one or more separate programs, each of which comprises an ordered listing of executable instructions for implementing logical functions. The software in the memory 42 includes a suitable operating system (O/S) 44, compiler 45, source code 46, and one or more applications 47 in accordance with exemplary embodiments.

The application 47 comprises numerous functional components such as computational units, logic, functional units, processes, operations, virtual entities, and/or modules.

The operating system 44 controls the execution of computer programs, and provides scheduling, input-output control, file and data management, memory management, and communication control and related services.

Application 47 may be a source program, executable program (object code), script, or any other entity comprising a set of instructions to be performed. When a source program, then the program is usually translated via a compiler (such as the compiler 45), assembler, interpreter, or the like, which may or may not be included within the memory 42, so as to operate properly in connection with the operating system 44. Furthermore, the application 47 can be written as an object oriented programming language, which has classes of data and methods, or a procedure programming language, which has routines, subroutines, and/or functions, for example but not limited to, C, C++, C#, Pascal, BASIC, API calls, HTML, XHTML, XML, ASP scripts, JavaScript, FORTRAN, COBOL, Perl, Java, ADA, .NET, and the like.

The I/O devices 43 may include input devices such as, for example but not limited to, a mouse, keyboard, scanner, microphone, camera, etc. Furthermore, the I/O devices 43 may also include output devices, for example but not limited to a printer, display, etc. Finally, the I/O devices 43 may further include devices that communicate both inputs and outputs, for instance but not limited to, a network interface controller (NIC) or modulator/demodulator (for accessing remote devices, other files, devices, systems, or a network), a radio frequency (RF) or other transceiver, a telephonic interface, a bridge, a router, etc. The I/O devices 43 also include components for communicating over various networks, such as the Internet or intranet.

When the computer 40 is in operation, the processor 41 is configured to execute software stored within the memory 42, to communicate data to and from the memory 42, and to generally control operations of the computer 40 pursuant to the software. The application 47 and the operating system 44 are read, in whole or in part, by the processor 41, perhaps buffered within the processor 41, and then executed.

When the application 47 is implemented in software it should be noted that the application 47 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage. Any reference signs in the claims should not be construed as limiting the scope.

1. A cardiac analysis system for use during a cardiac rehabilitation exercise, comprising:
a first input for receiving an indication of a heart rate of a user on cardiac medication;
a second input for receiving an indication of an activity level of the user;
a third input for receiving an indication of the respiration frequency of the user
a memory for storing thresholds for each of the activity level, the heart rate and the respiration frequency for a particular user, wherein the thresholds define ranges of activity level, heart rate and respiration frequency and
a controller for processing the indications received at the first to third inputs, comparing them with the thresholds, and determining whether or not the exercise being undertaken is suitable for cardiac rehabilitation; wherein the indication of the respiration frequency is used to correct the influence of the cardiac medication on the heart rate of the user for the determination that whether or not the exercise being undertaken is suitable for cardiac rehabilitation.

2. A system as claimed in claim 1, further comprising a fourth input for receiving an indication from the user of the user's feeling of the effort required by the exercise.

3. A system as claimed in claim 2, wherein the controller is adapted to update the thresholds in dependence on the fourth input.

4. A system as claimed in claim 1, wherein the controller is adapted to derive a Borg rating of perceived exertion score.

5. A system as claimed in claim 4, wherein the thresholds define four sub-categories of suitable exercise conditions corresponding to a Borg rating of perceived exertion score of 11, 12, 13 or 14.

6. A system as claimed in claim 1, wherein he controller is adapted to provide an output warning when the exercise is not determined to be suitable for cardiac rehabilitation.

7. A cardiac monitoring system comprising:
a cardiac analysis system as claimed in claim 1;
a heart rate monitor;
a respiration monitor; and
an activity level monitor.

8. A system as claimed in claim 7, wherein the activity level monitor comprises an accelerometer.

9. A cardiac analysis method for use during cardiac rehabilitation exercise of a subject, comprising:
storing thresholds for each of the activity level, the heart rate and the respiration frequency for a particular user on cardiac medication, wherein the thresholds define ranges of activity level, heart rate and respiration frequency
receiving an indication of a heart rate of the subject;
receiving an indication of an activity level of the subject;
receiving an indication of the respiration frequency of the subject and
processing the indications received by comparing them with the thresholds, and thereby determining whether or not the exercise being undertaken is suitable for cardiac rehabilitation; wherein the indication of the respiration frequency is used to correct the influence of the cardiac medication on the heart rate of the user for the determination that whether or not the exercise being undertaken is suitable for cardiac rehabilitation.

10. A method as claimed in claim 9, further comprising receiving an indication from the subject of the subject's own feeling of the effort required by the exercise.

11. A method as claimed in claim 10, wherein the controller is adapted to update the thresholds in dependence on the indication of the subject's own feeling of the effort required by the exercise.

12. A method as claimed in claim 9, comprising deriving a Borg rating of perceived exertion score.

13. A method as claimed in claim 12, wherein the thresholds define four sub-categories of suitable exercise conditions corresponding to a Borg rating of perceived exertion score of 11, 12, 13 or 14.

14. A method as claimed in claim 9, comprising providing an output warning when the exercise is not determined to be suitable for cardiac rehabilitation.

15. A computer program comprising code means which is adapted, when said program is run on a computer, to implement the method of claim 9.
